(19) United States
(12) Patent Application Publication
(10) Pub. No.: US 2010/0240023 A1
(43) Pub. Date: Sep. 23, 2010

(54) METHOD FOR EXTRACTING DEOXYRIBONUCLEIC ACIDS (DNA) FROM MICROORGANISMS POSSIBLY PRESENT IN A BLOOD SAMPLE

(75) Inventors: Jean-Pierre Hermet, Boulogne-Billancourt (FR); Isabelle Besson-Faure, Yerres (FR); Anne-Elodie Houlle-Declomesnil, Bretteville L'Orgueilleuse (FR)

Correspondence Address: ARENT FOX LLP, 1050 CONNECTICUT AVENUE, N.W., SUITE 400, WASHINGTON, DC, 20036, US

(73) Assignee: GeneOhm Sciences, Inc., San Diego, CA (US)

(21) Appl. No.: 12/301,437

(22) PCT Filed: May 18, 2007
(86) PCT No.: PCT/FR2007/051300

§ 371 Date: May 5, 2010

(30) Foreign Application Priority Data
May 19, 2006 (FR) 0604531

(51) Int. Cl. C12Q 1/70; C07H 1/06; C12Q 1/68

(52) U.S. Cl. 435/5; 536/23.1; 435/6

(57) ABSTRACT

The present invention relates to a method for extracting DNA from microorganisms possibly present in a blood sample, comprising the following steps: i) filtration of a blood sample through a filtration membrane, the pores of which have a diameter ranging from 0.01 μm to 50 μm, in particular from 0.1 μm to 10 μm, and most particularly from 0.2 μm to 1 μm; ii)) washing of said filtration membrane; and iii) extraction of the deoxyribonucleic acids from the microorganisms possibly present on said filtration membrane.

The present invention relates to the field of the detection and identification of any microorganisms present in a blood sample. It concerns particularly a method of extracting deoxyribonucleic acids (DNA) from any microorganisms present in a blood sample with a view to identification thereof.

The development of simple and rapid methods allowing the detection and/or identification and/or determination of the concentration of any microorganisms present in a blood sample is a major challenge in particular in the health field.

Currently, molecular biology methods and in particular polymerase chain reaction (PCR) techniques are expanding rapidly.

This is because these techniques present great sensitivity and specificity for the identification and/or quantification of microorganisms. However, the application of these molecular biology methods and particularly polymerase chain reaction (PCR) techniques to blood samples presents difficulties, in particular in terms of sensitivity and implementation.

This is because blood samples comprise agents inhibiting molecular biology techniques and in particular polymerase chain reaction (PCR) techniques.

These inhibiting agents may interfere with the DNA extraction methods and/or degrade the deoxyribonucleic acids (DNA) of the cells and/or inhibit the activity of the enzymes, in particular the polymerases used in various molecular biology techniques.

By way of examples of such agents inhibiting in particular polymerase chain reaction (PCR) techniques, in particular present in whole blood, calcium irons, haemoglobin, lactoferrin, haemin, urea and blood heparin can be cited (Al-Soud W A and Radstrom P, 2001. Purification and characterization of PCR-inhibitory components in blood cells. J Clin Microbiol 39:2:485-493). There therefore remains a need for methods of extracting DNA from microorganisms present in a blood sample, with a view to detection and/or identification thereof.

The inventors have discovered a particular method for wholly or partially resolving the problems mentioned above.

According to a first aspect, an object of the invention is a method of extracting DNA from any microorganisms present in a blood sample, comprising the following steps:

i) the filtration of a blood sample through a filtration membrane whose pores have a diameter ranging from 0.01 μm to 50 μm, in particular from 01 μm to 10 μm and especially from 0.2 μm to 1 μm;
ii) washing of the said filtration membrane;
iii) extraction of the deoxyribonucleic acids from any microorganisms present on the said filtration membrane.

“Blood sample”, within the meaning of the present invention, means a whole blood or haemoculture sample that has possibly been treated with a view to reducing the level of and/or eliminating the red corpuscles and/or platelets present in the said sample.

The step of treating a blood sample with a view to reducing the level of and/or eliminating the red corpuscles and/or the platelets present in the said sample can be implemented according to techniques well known to persons skilled in the art.

By way of examples of such techniques, those described in the PCT application WO 03/025207 can be cited:
the platelet aggregation technique using aggregation agents such as specific antibodies of a platelet antigen, followed by the filtration of the treated sample;
the technique of agglutination of red corpuscles using aggregation agents such as lectins followed by the filtration of the treated sample.

Whole-blood samples can be obtained according to techniques well known to persons skilled in the art using for example a needle fitted with a syringe introduced in particular into a vein of the forearm or the bend of the elbow of an individual. A sample of 1 to 10 ml of blood taken in particular on an anticoagulant, in particular EDTA, sodium citrate or heparin, obtained from a human or animal subject, can be sufficient to implement the method according to the present invention.

Haemoculture blood samples can be obtained after seeding of whole blood taken from a human or animal subject on culture media appropriate to the development of the microorganisms.

A filtration membrane adapted for the method according to the invention can be identified simply by a person skilled in the art in the light of his general knowledge.

In particular, the filtration membrane according to the invention can be chosen from the group comprising membranes made from polyvinylidene fluoride, polyester, nylon, polypropylene, polycarbonate and polyethersulfone, in particular polyvinylidene fluoride.

Preferably, the said filtration membrane is not based on cellulose.

The filtration step i) of the method according to the invention can be carried out using devices and filter supports well known to persons skilled in the art, in particular a support as described in patent application US 2004/0208796.

At step ii), “washing” means a step for reducing the level of impurities retained on the filtration membrane whilst allowing at least some of the microorganisms to be held on the said membrane.

The impurities may in particular be agents inhibiting molecular biology techniques (Wilson J G 1997. Inhibition and Facilitation of Nucleic Acid Amplification Appl Environ Microbiol 63:10:3741-3751) in particular:
plasma proteins, immunoglobulins G (Al-Soud, W. A., Jonsson, L. J., and Radstrom, P. 2000. Identification and characterization of immunoglobulin G in blood as a major inhibitor of diagnostic PCR. J Clin Microbiol 38:1:345-350);
enzymes (proteases);
polyamines;
sodium polyanethol, sulfonate (SPS);
blood anticoagulants such as heparin (Satsangi J., Jewell D. P., Welsh K., Bunce M., and Bell J. I. 1994. Effect of heparin on polymerase chain reaction. Lancet 343:8911:1509-1510) and ethylene diamine tetracetic acid (EDTA) (Al-Saud W. A. and Radstron P. 2001. Purification and characterization of PRC-inhibitory components in blood cells. J Clin Microbial 39:2:485-493);
haemoglobin, hemin (Akane A., Matsubara K., Nakamura H., Tahahashi S., and Kimura K. 1994. Identification of the heme compound copurified with deoxyribonucleic acid (DNA) from bloodstains, a major inhibitor of polymerase chain reaction (PCR) amplification. J Forensic Sci 39:2:362-372), bilirubin, phenols;
detergents such as sodium dodecyl sulfate or tryton X100; and
bile acids.

Advantageously, the said step ii) also makes it possible to lyse, in particular by means of a hypothermic shock, the red corpuscles of the blood sample and in particular to eliminate their content, in particular the haemoglobin contained in these red corpuscles.

A person skilled in the art would be able to determine simply the washing solution volume used at step ii) in the light of his general knowledge.

The washing solution volume may correspond to a volume of between ¼ and 20, and particularly between k and 10 and especially between 1 and 5 times the volume of the blood sample filtered at step i).

For example, when the blood sample filtered at step i) has a volume ranging from 0.2 to 5 ml and especially from 0.5 to 1 ml, then the washing step ii) can be carried out with a washing solution volume ranging from 1 to 10 ml, in particular from 3 to 5 ml and especially 3 ml.

By way of example again, when the blood sample filtered at step i) has a volume ranging from 5 to 100 ml, especially from 5 to 20 ml, then the washing step ii) can be carried out with a washing solution with a volume ranging from 5 to 50 ml, in particular from 8 to 20 ml and especially 10 ml.

The washing solution adapted for the method according to the invention can be identified simply by a person skilled in the art in the light of his general knowledge.

The washing solution can be chosen in particular from aqueous solutions, in particular water and especially osmosed water, preferably sterilised by filtration.

The said sterilisation of the water by filtration can be carried out in particular using a filtration membrane, the pores of which may have a diameter of approximately 0.22 μm.

By way of examples of washing solutions that can be used for the washing step ii) according to the invention, pure water for molecular biology available from Eppendorf with in particular the reference 0032.006.159, pure water coming from known purification systems with in particular the Purelab® system from ELGA Labwater, or the Milli-Q® system from Millipore, or the Infinity UV/UF® system from Werner, can be cited.

When the method according to the invention uses a filter device and/or support, the washing step ii) can be performed in particular by passing the washing solution over the said filtration membrane, leaving the filtration membrane in place in the filter device and/or on the filter support used at step i).

The step of extraction of deoxyribonucleic acids of any microorganisms present on the said filtration membrane can be carried out according to techniques well known to the persons skilled in the art such as physical lysis methods, in particular thermal or by sonication, chemical lysis methods, in particular described in the manual of Sambrook J., Fritsch E. F. and Maniatis T. (Molecular cloning: a laboratory manual, 2^{nd} ed. Cold Spring Harbor Laboratory Press, Cold Spring, N.Y., 1989).

“Microorganism” means a living organism belonging to one of the following three kingdoms, that of monera, protists and protozoa. Microorganisms have a eukaryotic or prokaryotic or akaryotic cellular structure, a microscopic or ultramicroscopic size and are single cell. By way of examples of microorganisms, the following can be cited:
bacteria such as Escherichia coli, klebsiella, shigella, streptococci, staphylococci, enterococci, proteus, enterobacter, serratia, pseudomonas, bacillus, corynebacteria, listeria, acinetobacter, cryptococci, bartonella and microbacteria; and
fungi such as candida and aspergillus.

In particular, the method according to the invention also comprises the following step:

iv) the identification of the microorganisms, in particular bacteria, viruses, protozoa and/or fungi, possibly present in the said blood sample.

“Identification” means the determination of the species of a microorganism.

The identification of the microorganisms can be carried out using deoxyribonucleic acids extracted from the said microorganisms by molecular biology techniques well known to persons skilled in the art.

In particular, step iv) comprises the use of a technique using an activity of the polymerase type chosen from the group comprising end-point polymerase chain reaction, multiplex polymerase chain reaction, qualitative polymerase chain reaction, semi-quantitative polymerase chain reaction and quantitative polymerase chain reaction.

End-point PCR equipment available in particular from Applied Biosystems under the name “ABI PRISM®” and from Roche Diagnostics under the name “COBAS Amplicor®” can be used in the method according to the invention.

Real-time PCR equipment available in particular from Applied Biosystems under the name “7500 Real-time PCR System®”, from Roche Diagnostics under the name “CODAS Taqman®” and from Genesystems under the name “GeneDisc Cycler®” can be used in the method according to the invention.

Real-time PCR kits with pairs of initiators and specific sensors for a microorganism available from Roche with the “Lightcycler Septifast Kit®” under the references 04469046001 or 04488814001); from Biotage with the “microbial species determination and resistance” kits under references 8, 7 and 12; from BAG (Biologish Analysensystem GmbH) with the kits “Hyplex StaphyloResist®” under the reference 3801, “Hyplex StaphyloResist Plus” under the reference 3809 and “Hyplex EnteroResist®” under the reference 3802 can be used in the method according to the invention. For example the real-time PCR kit available from Argène with the reference 69-002 can be used for identifying and determining the level of Epstein-Barr virus (EBV) present in a blood sample.

According to a particular embodiment, the method according to the invention also comprises the following step:

v) the identification of at least one antibiotic resistance gene in at least one microorganism possible present in the said blood sample.

The identification of a antibiotic resistance gene using deoxyribonucleic acids extracted from microorganisms can be made by molecular biology techniques well known to persons skilled in the art.

In particular, step v) comprises the use of a polymerase chain reaction technique with in particular pairs of initiators and in particular specific sensors for at least one antibiotic resistance gene.

According to another particular embodiment, the method according to the invention also comprises the following step:

vi) the determination of the level of microorganisms, in particular bacteria, viruses, protozoa and/or fungi possibly present in the said blood sample.

“Level of microorganisms” means the quantity of microorganisms present in the blood sample on which the method according to the invention is implemented.

The determination of the level of microorganisms possibly present in the said blood sample can be carried out by techniques well known to persons skilled in the art.

By way of example, this determination can be carried out by comparing the results obtained at the microorganism identification step iv) with the results obtained with positive references corresponding to given dilutions of the said microorganisms.

In particular, step vi) comprises the use of the real-time polymerase chain reaction technique.

Advantageously, at least one of steps iv), v) or vi) of the method according to the invention is performed in a medium adapted to at least one molecular biology technique, comprising an extract of the deoxyribonucleic acids of the microorganisms obtained at step iii).

“Medium adapted to at least one molecular biology technique” means a medium comprising agents for increasing the efficiency and/or sensitivity and/or specificity of these techniques, in particular polymerase chain reactions.

Such media are well known to persons skilled in the art, as described in the literature (Wilson, J. G. 1997, Inhibition and Facilitation of Nucleic Acid Amplification. Appl Environ Microbiol 63:10:3741-3751).

By way of example of media adapted to PCR techniques, the following can be cited: media comprising in particular bovine serum albumin (Akane A., Matsubara K., Nakamura H., Tahahashi S., and Kimura K. 1994. Identification of the heme compound copurified with deoxyribonucleic acid (DNA) from bloodstains, a major inhibitor of polymerase chain reaction (PCR) amplification. J Forensic Sci 39:2:362-372), glycerol, magnesium ions (Satsangi J., Jewell D. P. Welsh K., Bunce M., and Bell J. I. 1994. Effect of heparin on polymerase chain reaction. Lancet 343:8911:1509-1510) trimethyl glycine, dimethylsulfoxide, polyethylene glycol, Tween 20®, Tween 40®, Tween 80®, DMSO, Triton X-100®, Triton X-114®, betaine monohydrate, betaine trimethylglycine, PEG 35000, PEG 400, PEG 6000 and acetamide.

According to a particular embodiment, the method according to the invention comprises, prior to step i), the following steps:

a) the addition to the whole blood or haemoculture of a red corpuscle agglutination solution and/or a platelet aggregation solution; and
b) the filtration of the preparation obtained at step a) through a filter whose pores have a diameter ranging from 2 μm to 50 μm, in particular ranging from 10 μm to 25 μm and especially 17 μm.

In particular, the said red corpuscle agglutination solution comprises at least one agglutination agent chosen from the group comprising lectins, polyethylenimine, polyvinylpyrrolidone, gelatines, dextrans and polyethylene glycols, in particular lectins.

In particular, the lectins are chosen from the group comprising lens culinaris, Phaseolus vulgaris, Vicia sativa, Vicia faba and Erythrina corallodendron lectins.

The agglutination agent, in particular lectin, can be present in the agglutination solution at a concentration ranging from 10 μg/ml to 200 μg/ml, in particular from 15 μg/ml to 100 μg/ml and especially from 20 μg/ml to 30 μg/ml.

In particular, the said platelet aggregation solution comprises at least one platelet aggregation agent chosen from the group comprising specific platelet antigen antibodies, thrombin, trypsin, collagen, thromboxane A2, the platelet activation factor, adrenalin, arachidonic acid, serotonin and epinephrine, in particular the specific antibodies of a platelet antigen and collagen.

The specific antibodies of a platelet antigen can be present in the platelet aggregation solution at a concentration ranging from 0.5 μg/ml to 100 μg/ml, in particular from 1 μg/ml to 60 μg/ml and especially from 5 μg/ml to 45 g/ml.

The collagen can be present in the platelet aggregation solution at a concentration ranging from 0.05 μg/ml to 50 μg/ml, in particular from 1 μg/ml to 20 μg/ml.

Other advantages and characteristics of the invention will emerge in the light of the following figures and examples.

The following figures and examples are given by way of illustration and non-limitatively:

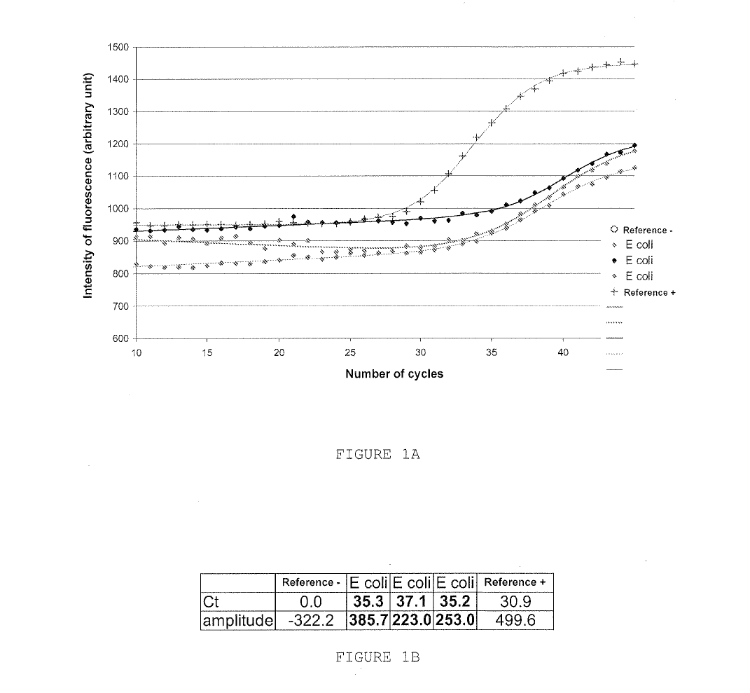

FIGS. 1 (A and B) illustrates the identification and quantification of Escherichia coli present in whole blood samples. FIG. 1A illustrates in the form of a curve the results of real-time polymerase chain reactions using a fluorescent probe and a pair of specific Escherichia coli primers. FIG. 1B illustrates in the form of a table the threshold cycle and the amplitude of the real-time polymerase chain reactions.

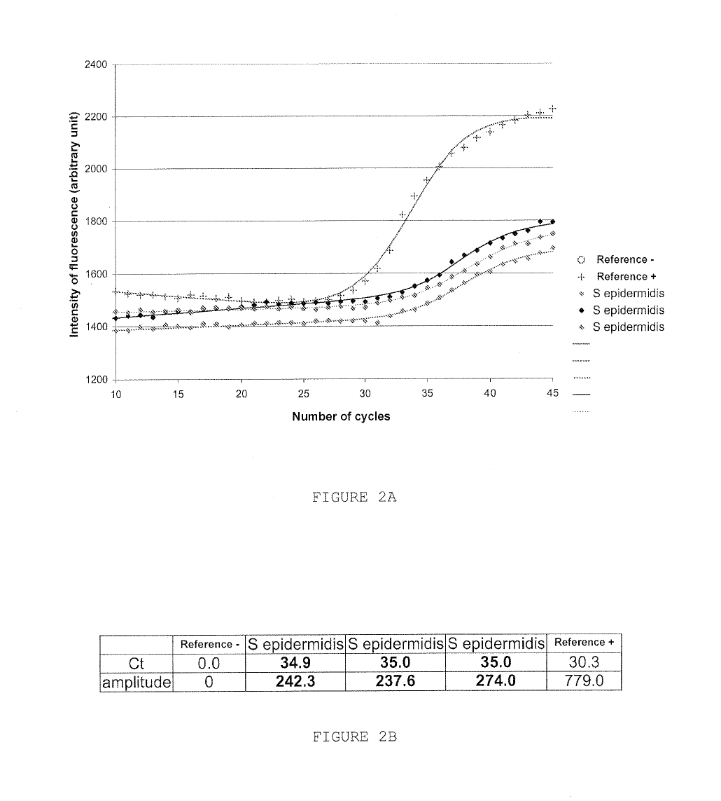

FIGS. 2 (A and B) illustrates the identification and quantification of Staphylococcus epidermis present in whole blood samples. FIG. 2A illustrates in the form of a curve the results of the real-time polymerase chain reactions using a fluorescent probe and a pair of specific Staphylococcus epidermis primers. FIG. 2B illustrates in the form of a table the threshold cycle and the amplitude of the real-time polymerase chain reactions.

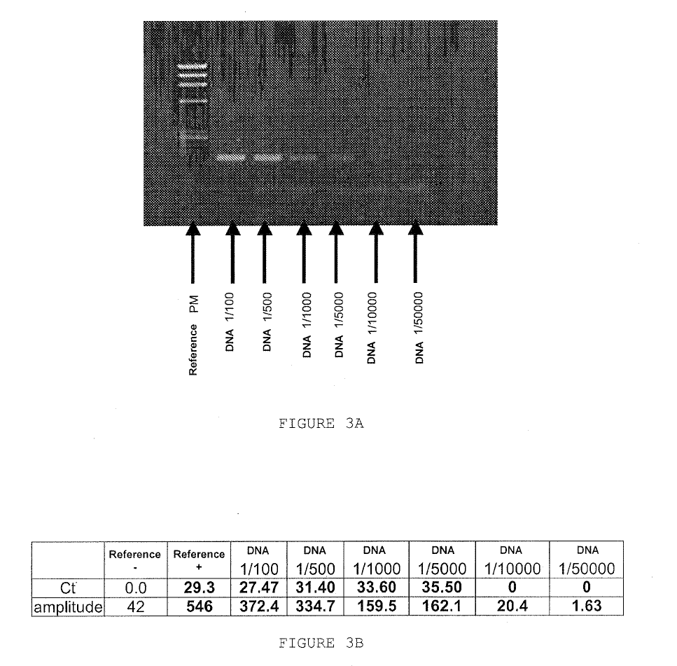

FIG. 3A shows a photograph of an agarose gel after end-point polymerase chain reaction (PCR) and migration using a fluorescent probe and a pair of specific Escherichia coli primers.

FIG. 3B shows the results of the real-time quantitative polymerase chain reactions (PCR) using a fluorescent probe and pair of specific Escherichia coli primers.

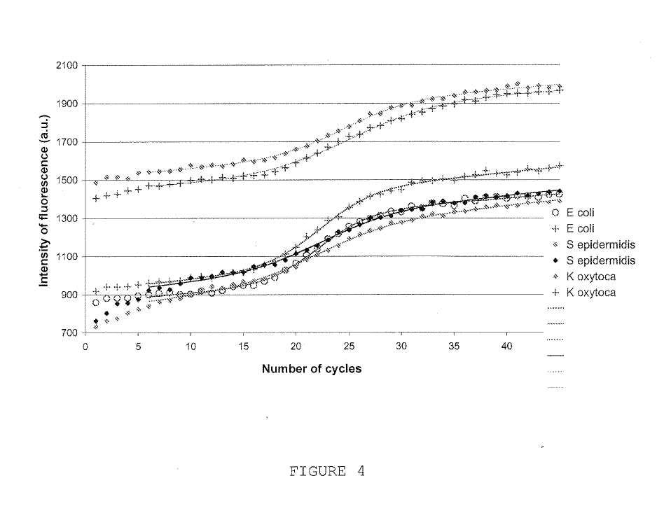

FIG. 4 illustrates in the form of curves the identification and quantification of Escherichia coli, staphylococcus epidermis and Klebsiella oxytoca present in haemoculture blood samples. FIG. 4 illustrates in the form of a curve the results of the real-time polymerase chain reactions using a fluorescent probe and a pair of specific Escherichia coli, staphylococcus epidermis and Klebsiella oxytoca primers.

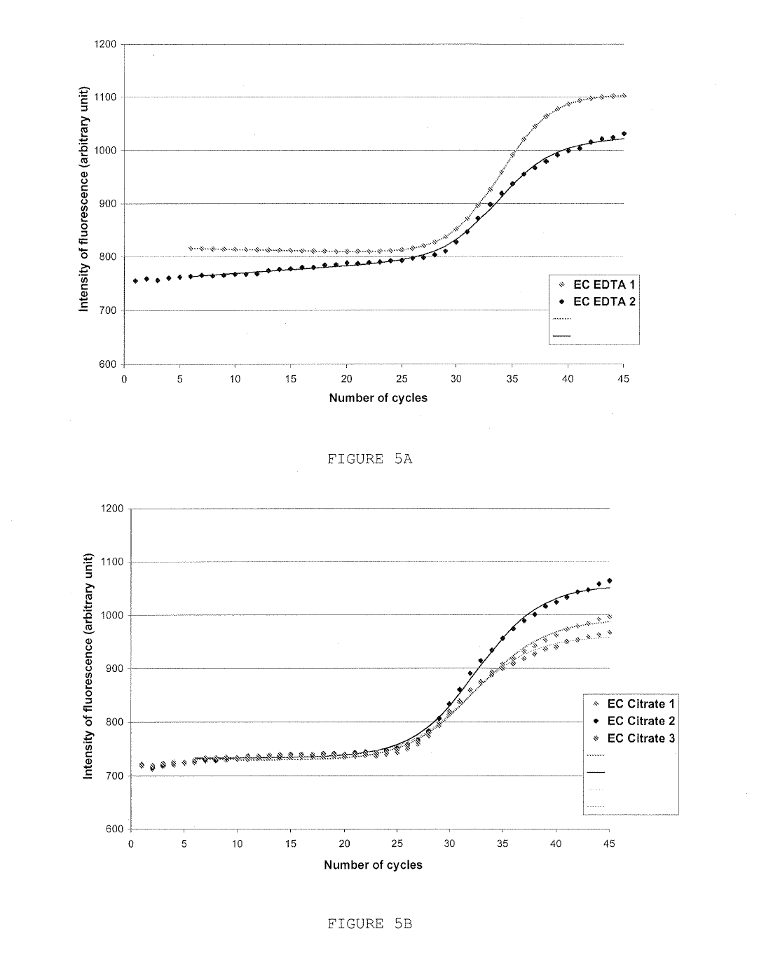

FIG. 5 illustrates in the form of curves the identification and quantification of Escherichia coli present in whole blood samples taken on EDTA (FIG. 5A) and sodium citrate (FIG. 5B) and of Staphylococcus epidermis present in whole blood samples on heparin (FIG. 5C). FIG. 5 illustrates in the form of a curve the results of the real-time polymerase chain reactions using a fluorescent probe and a specific Escherichia coli and staphylococcus epidermis primers.

### EXAMPLES

### I. Example I

### Method of Preparing Blood Samples

### I.1 Method of Preparing Haemoculture Blood Samples

100 μl of haemoculture blood sample was taken using a sterile syringe through the septum of haemoculture flasks. 1 ml of filtered osmosed water (using a filter whose pores have a diameter of approximately 0.22 μm) was then added to each sample.

Each sample was filtered through a polyvinylidene fluoride filtration membrane with a diameter of 25 to 32 mm whose pores have a diameter ranging from 0.2 μm to 1 μm. The filtration membrane was contained in a filter support as described in the patent application US 2004/0208769.

Each filtration membrane was then washed with 1 to 10 ml of pure or osmosed water, by filtration using a filter whose pores have a diameter of approximately 0.22 μm.

After filtration, each filtration membrane was recovered by means of sterile tweezers and inserted in a sterile microtube containing 200 μl to 1 ml of:
pure water for molecular biology such as the one marketed by Eppendorf, or
pure water for molecular biology with bovine serum albumin (BSA) added to the extent of 0.7%, or
pure water for molecular biology with Tris HCl added at 125 mM, betaine at 160 mM and dimethylsulphoxide (DMSO) at 5%, at a pH of 9.3 adjusted with 1 M NaOH.

### I.1 Method of Preparing Whole Blood Samples

5 ml of whole blood sampled on anticoagulant (EDTA, heparin or sodium citrate) was added to 20 ml of red corpuscle agglutination solution.

The said agglutination solution comprising lens culinaris lectin at 25 μg/ml, polyethylene glycol (PEG) at 1% in a medium containing 75% brain heart broth and 25% Tryptone Soy Broth (TSB).

After incubation for 30 minutes at room temperature, the red corpuscles are agglutinated in a concentrate.

15 to 20 ml of red corpuscle concentrate supernatant was taken off and incubated along with 1 ml of platelet aggregation solution.

The said aggregation solution comprising the anti-CD9 monoclonal antibody at 45 μg/ml in a medium containing 75% brain heart broth and 25% TSB.

After aggregation of the platelets, the preparation was filtered through a filter whose pores have a diameter of approximately 17 μm.

This filtration step made it possible to retain on the filter the platelet aggregates and the blood cells with a size greater than the size of the pores of the filter.

The filtrate was then once again filtered through a polyvinylidene fluoride filtration membrane with a diameter from 25 to 32 mm whose pores have a diameter ranging from 0.2 μm to 1 μm. The filtration membrane was contained in a filter support as described in the patent application US 2004/0208796.

The filtration membrane was then washed with 8 to 20 ml of pure or osmosed water, by filtration.

After filtration, the filtration membrane was recovered by means of sterile tweezers and inserted in a sterile microtube containing between 200 μl and 1 ml of either:
pure water for molecular biology such as the one marketed by Eppendorf, or
pure water for molecular biology with bovine serum albumin (BSA) added to the extent of 0.7%, or
pure water for molecular biology with Tris HCl added at 125 mm, betaine at 160 mm and dimethylsulphoxide (DMSO) at 5%, at a pH of 9.3 adjusted with 1 M NaOH.

### II. Example II

### Identification of Microorganisms Present in Blood Samples

### II.1 Method of Extracting DNA

DNA was extracted from filtration membranes contained in sterile microtubes, obtained after the implementation of the methods described in example 1.

Each microtube was subjected to a succession of heating and/or sonication steps, and/or freezing as described in Maniatis (Sambrook, J., Fritsch, E. F. and Maniatis, T. in “Molecular Cloning” (1992), Cold Spring Harbor Laboratory Press, Cold Spring Harbor, N.Y.).

### II.2 Polymerase Chain Reactions (PCR) Techniques Used

### II.2.1 Real-Time Quantitative Polymerase Chain Reaction (PCR) Techniques

30 to 42 μl of DNA extracted according to the method described in the previous section (II.1) was added to 30 μl of mixture containing Taq® polymerase and deoxyribonucleotide triphosphates (dNTP) in a buffer medium.

12 μl of this preparation was deposited in wells containing the fluorescent probe and the pair of specific primers of the microorganism strains to be detected such as those described in the PCT application WO 2004/024944. The negative reference corresponds to a well with no fluorescent probe.

The positive reference corresponds to a well comprising a synthetic sequence of DNA with the fluorescent probes and the pair of specific primers of the said sequence.

The results have been expressed firstly in the form of amplification curves showing the variation in intensity of the fluorescence as a function of the amplification cycles (curves presented in FIGS. 1, 2 and 4).

Secondly, the tables in FIGS. 1 and 3 present, for each polymerase chain reaction performed:
the threshold cycle or Ct, corresponding to the minimum of cycles necessary for achieving the exponential DNA amplification phase;
the amplitude corresponding to the difference between the maximum and minimum fluorescence intensity obtained.
   II.2.2 Conventional Polymerase Chain Reaction (PCR) Techniques and then Migration on Agarose Gel

7 μl of DNA extracted according to the method described in the previous section (II.1) were added to 43 μl of a mixture containing Taq® polymerase, deoxyribonucleotide triphosphates (dNTP) and the pair of specific primers for the microorganisms to be detected, in a buffer medium.

40 amplification cycles were performed.

Then 15 μl of amplified DNA was pipetted, 3 μl of bromophenol blue was added thereto and this preparation was deposited in a well within an agarose gel.

A current was applied in order to make the amplified DNA fragments migrate according to their molecular weight (MW).

A photograph of an agarose gel after migration is presented in FIG. 3A.

II.3 Identification of Escherichia coli and Staphylococcus epidermis Present in Whole Blood Samples

5 ml of whole blood was treated according to the protocol described in paragraph I.2 in which the filtration membrane was made from polyvinylidene fluoride 25 mm in diameter, the pores of which have a diameter of approximately 0.65 μm. 8 ml of osmosed water filtered using a filter whose pores have a diameter of approximately 0.22 mm was used to wash the filtration membrane.

After extraction of the DNA according to the protocol described in section II.1, real-time polymerase chain reactions (PCR) were performed according to the protocol described in section II.2.1, using a fluorescent probe and a pair of specific primers of Escherichia coli and Staphylococcus epidermis.

FIGS. 1 (A and B) shows the results of the polymerase chain reactions using fluorescent probes and a pair of specific primers of Escherichia coli.

FIGS. 2 (A and B) shows the results of the polymerase chain reactions using fluorescent probes and a pair of specific primers of Staphylococcus epidermis.

These results show that the method of preparing a whole blood sample according to the invention makes it possible to obtain samples in which the microorganisms can be identified and quantified in a specific and reproducible manner by polymerase chain reaction techniques.

II.4 Identification of Escherichia coli Present in Whole Blood Samples

5 ml of whole blood was treated according to the protocol described in section I.2, in which the filtration membrane was made from polyvinylidene fluoride at 25 mm in diameter and the pores of which have a diameter of approximately 0.65 μm. 8 ml of osmosed water filtered by means of a filter whose pores have a diameter of approximately 0.22 μm was used to wash the filtration membrane.

After extraction of the DNA according to the protocol described in section II.1, dilutions of the DNA extract in pure water for molecular biology were carried out in order to test the sensitivity of the PCRs: dilution of the DNA extract from 1/100 to 1/50000.

Real-time quantitative polymerase chain reactions (PCRs according to the protocol described in section II.2.1 and in end point according to the protocol described in section II.2.2 were then performed. A fluorescent probe and a pair of specific primers of Escherichia coli were used.

FIG. 3A shows a photograph of an agarose gel after conventional polymerase chain reaction (PCR) and migration using a fluorescent probe and a pair of specific primers of Escherichia coli.

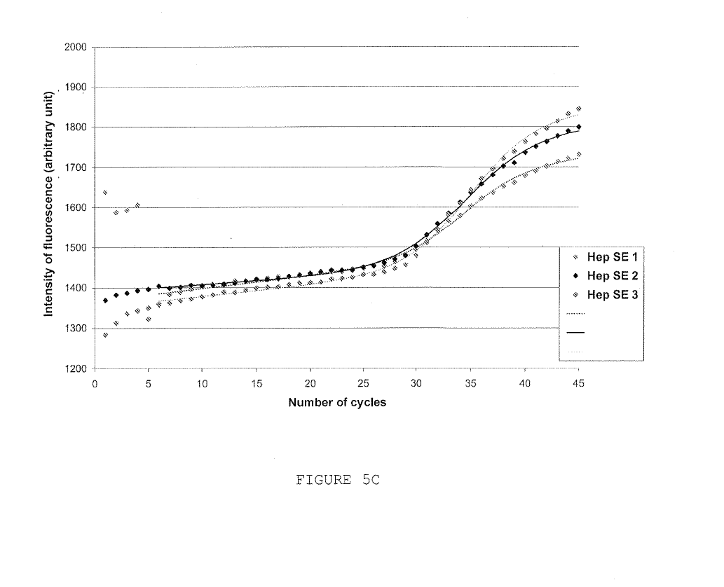

FIG. 35 shows the results of the real-time quantitative polymerase chain reactions (PCRs) using fluorescent probes and a pair of specific probes of Escherichia coli.

These results show that the method of preparing a blood sample issuing from whole blood according to the invention makes it possible to obtain samples in which the microorganisms can be identified and quantified in a specific and reproducible manner and with a certain sensitivity by real-time or end point polymerase chain reaction techniques.

II.5 Identification of Escherichia coli, Staphylococcus epidermis and Klebsiella oxytoca Present in Haemoculture Blood Samples

Haemoculture blood samples were prepared according to the protocol described in section I.1 in which the filtration membrane was made from polyvinylidene fluoride 25 mm in diameter and where the diameter of the pores is approximately 6.65 μm. 3 ml of osmosed water was used to wash the filtration membrane.

After extraction of the DNA according to the protocol described in section II.1, real-time quantitative polymerase chain reactions (PCRs) were performed according to the protocol described in section II.2.1 using a fluorescent probe and a pair of specific primers of Escherichia coli, Staphylococcus epidermis and Klebsiella oxytoca. The results are presented in FIG. 4.

These results show that the method of preparing a blood sample issuing from haemoculture according to the invention makes it possible to obtain samples in which the microorganisms can be identified and quantified in a specific and reproducible manner by polymerase chain reaction techniques.

II.6 Identification of Escherichia coli and Staphylococcus epidermis Present in Whole Blood Samples Taken from Different Anticoagulants

5 ml of whole blood taken from tubes containing a blood anticoagulant of the tripotassic EDTA, sodium citrate or heparin (lithium heparin) type was treated according to the protocol described in section I.2 in which the filtration membrane was made from 25 mm diameter polyvinylidene fluoride and the pores of which have a diameter of approximately 0.65 μm. 8 ml of osmosed water filtered by means of filter whose pores have a diameter of approximately 0.22 μm was used to wash the filtration membrane.

After extraction of the DNA according to the protocol described in section II.1, real-time polymerase chain reactions (PCRs) were carried out according to the protocol described in section II.2.1, using a fluorescent probe and a pair of specific primers of Escherichia coli and Staphylococcus epidermis.

FIGS. 5A and 5B show the results of the polymerase chain reactions using fluorescent probes and a pair of specific primers of Escherichia coli for bacterial DNA issuing from whole blood sampled on tripotassic EDTA (FIG. 5A) and sodium citrate (FIG. 5B).

FIG. 5C shows the results of the polymerase chain reactions using fluorescent probes and pair of specific primers of Staphylococcus epidermis for bacterial DNA issuing from whole blood sampled on heparin (FIG. 5C).

These results show that the method of preparing a blood sample issuing from whole blood sampled on different coagulants according to the invention makes it possible to obtain samples in which the microorganisms can be identified and quantified in a specific and reproducible manner by polymerase chain reaction techniques.

1. A method of extracting DNA from any microorganisms present in a blood sample comprising the following steps:
i) the filtration of a blood sample through a filtration membrane whose pores have a diameter ranging from 0.01 μm to 50 μm;
ii) the washing of said filtration membrane; and
iii) the extraction of the deoxyribonucleic acids from any microorganisms present on the said filtration membrane.

2. A method according to claim 1, characterised in that it also comprises the following step:
iv) identification of the microorganisms possibly present in the said blood sample.

3. A method according to claim 2, characterised in that step iv) comprises the use of molecular biology technique using an activity of the polymerase type chosen from the group comprising end point polymerase chain reaction, real-time polymerase chain reaction, multiplex polymerase chain reaction, qualitative polymerase chain reaction, semi quantitative polymerase chain reaction and quantitative polymerase chain reaction.

4. A method according to any one of claims 1 to 3, characterised in that it also comprises the following step:
v) the identification of at least one resistance gene to an antibiotic in at least one microorganism possibly present in the said blood sample.

5. A method according to claim 4, characterised in that step v) comprises the use of a polymerase chain reaction technique.

6. A method according to claim 1 or 2, characterised in that it also comprises the following step:
vi) the determination of the level of microorganisms, in particular bacteria, viruses, protozoa and/or fungi possibly present in the said blood sample.

7. A method according to claim 1 or 2, characterised in that the said filtration membrane is chosen from the group comprising membranes made from polyvinylidene fluoride, polyester, nylon, polypropylene, polycarbonate and polyethersulfone, in particular polyvinylidene fluoride.

8. A method according to claim 1 or 2, characterised in that the said filtration membrane is not based on cellulose.

9. A method according to claim 1 or 2, characterised in that the said blood sample is chosen from the group comprising:
a whole blood sample: and
a haemoculture blood sample.

10. A method according to claim 1 or 2, characterised in that it comprises, prior to step i), the following steps:
a) the addition to the whole blood or to the haemoculture of an agglutination solution of red corpuscles and/or a platelet aggregation solution; and
b) the filtration of the preparation obtained at step a) through a filter whose pores have a diameter ranging from 2 μm to 50 μm.

11. A method according to claim 10, characterised in that the said agglutination solution comprises at least one agglutination agent chosen from the group comprising lectins, polyethylenimine, polyvinylpyrrolidone, gelatines, dextrans and polyethylene glycols.

12. A method according to one of claim 10, characterised in that the said platelet aggregation solution comprises at least one platelet aggregation agent chosen from the group comprising specific antibodies of a platelet antigen, thrombin, trypsin, collagen, thromboxane A2, the platelet activation factor, adrenalin, arachidonic acid, serotonin and epinephrine.
